(19) United States
(12) Patent Application Publication
(10) Pub. No.: US 2015/0157285 A1
(43) Pub. Date: Jun. 11, 2015

(54) X-RAY DIAGNOSIS APPARATUS

(71) Applicants: Kabushiki Kaisha Toshiba, Minato-ku (JP); Toshiba Medical Systems Corporation, Otawara-shi (JP)

(72) Inventors: Tadaharu KOBAYASHI, Otawara (JP); Shingo ABE, Nasushiobara (JP)

(73) Assignees: Kabushiki Kaisha Toshiba, Minato-ku (JP); Toshiba Medical Systems Corporation, Otawara-shi (JP)

(21) Appl. No.: 14/556,840

(22) Filed: Dec. 1, 2014

(30) Foreign Application Priority Data
Dec. 6, 2013 (JP) 2013-253497

(51) Int. Cl. A61B 6/00

(52) U.S. Cl. CPC A61B 6/467; A61B 6/487

(57) ABSTRACT

According to an embodiment, in an X-ray diagnosis apparatus, an X-ray detector detects X-rays having emitted from an X-ray irradiator and passed through the subject. A first input unit is arranged so that it can be pedaled, and is used for inputting first operational information in response to a pedaling operation by an operator. A second input unit is positioned apart from the first input unit so that the operator can touch the second input unit while touching an upper surface of the first input unit that faces away from a floor surface of a room, and is used for inputting second operational information in response to an operation by the operator. A controller performs control for changing the first and the second operational information or either thereof on the basis of operational information input by at least one of the first and the second input units.

### CROSS-REFERENCE TO RELATED APPLICATIONS

This application is based upon and claims the benefit of priority from Japanese Patent Application No. 2013-253497, filed on Dec. 6, 2013; the entire contents of which are incorporated herein by reference.

### FIELD

Embodiments described herein relate generally to an X-ray diagnosis apparatus that assists treatment using a device such as a catheter.

### BACKGROUND

In recent years, improvements have been achieved in X-ray diagnosis apparatuses, particularly in the cardiovascular field, in line with technical advancement in angiographic procedures using catheters and in interventional radiology (IVR). Such an X-ray diagnosis apparatus has a footswitch placed on a floor so that an operator can perform operations of, for example, enabling and disabling X-ray emission by one foot while operating a device such as a catheter by the hands. The footswitch includes a plurality of pedals for fluoroscopy and for photography. When concentrating on a patient, however, the operator cannot see the footswitch, and hence needs to take great care so as not to kick off a footswitch to be operated and so as not to commit errors in operation by pressing down a wrong pedal.

### BRIEF DESCRIPTION OF THE DRAWINGS

FIG. 1 is a block diagram illustrating the configuration of an X-ray diagnosis apparatus according to a first embodiment;

FIG. 2A and FIG. 2B are views illustrating the configuration of a second operation unit according to the first embodiment;

FIG. 3A and FIG. 3B are views illustrating examples of operations performed on the second operation unit according to the first embodiment;

FIG. 4 is a block diagram illustrating the configuration of an X-ray diagnosis apparatus according to a second embodiment;

FIG. 5A and FIG. 5B are views illustrating the configuration of a second operation unit according to the second embodiment;

FIG. 6A and FIG. 68 are views illustrating one example of an operation performed on the second operation unit according to the second embodiment;

FIG. 7 is a block diagram illustrating the configuration of an X-ray diagnosis apparatus according to a third embodiment;

FIG. 8A and FIG. 8B are views illustrating the configuration of a second operation unit according to the third embodiment;

FIG. 9A and FIG. 9B are views illustrating one example of an operation performed on the second operation unit according to the third embodiment;

FIG. 10 is a block diagram illustrating the configuration of an X-ray diagnosis apparatus according to a fourth embodiment;

FIG. 11A and FIG. 11B are views illustrating the configuration of a second operation unit according to the fourth embodiment; and

FIG. 12A and FIG. 12B are views illustrating one example of an operation performed on the second operation unit according to the fourth embodiment.

### DETAILED DESCRIPTION

According to embodiment, an X-ray diagnosis apparatus comprising, an X-ray irradiator, an X-ray detector, a first input unit, a second input unit and a controller. The X-ray irradiator that emits X-rays to a subject. The X-ray detector that detects X-rays emitted from the X-ray irradiator and having passed through the subject. The first input unit arranged in a manner that allows the first input unit to be pedaled, the first input unit being used to input first operational information in response to a pedaling operation performed thereon by an operator. The second input unit arranged apart from the first input unit at a position that allows the operator to touch the second input unit while touching an upper surface of the first input unit that faces away from a floor surface of a room, the second input unit being used to input second operational information in response to an operation performed thereon by the operator. The controller that performs control for changing the first and the second operational information or either thereof on the basis of operational information input by at least one of the first and the second input units. The following describes embodiments with reference to the drawings.

### First Embodiment

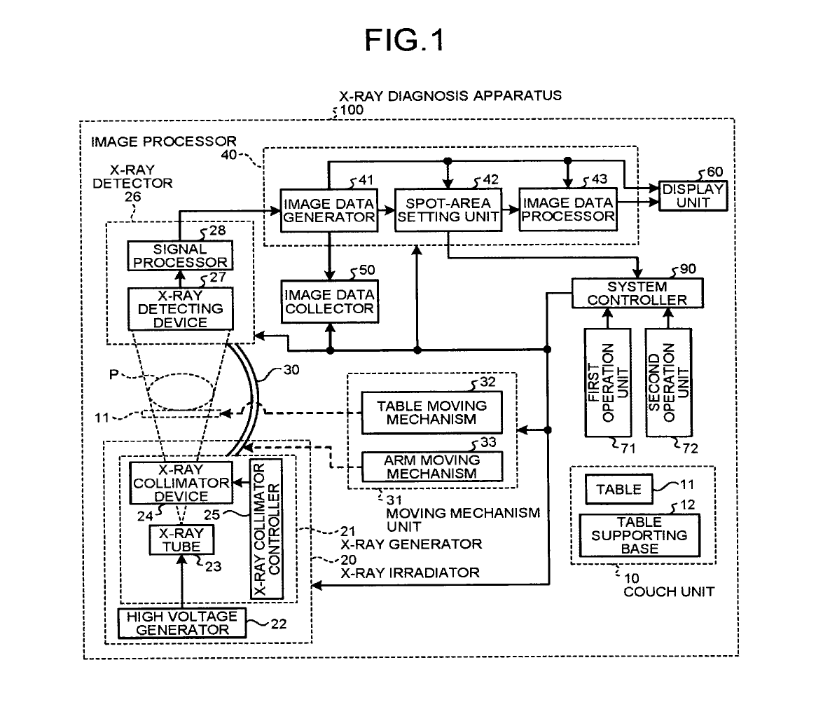

FIG. 1 is a block diagram illustrating the configuration of an X-ray diagnosis apparatus according to a first embodiment. This X-ray diagnosis apparatus 100 includes a couch unit 10, an X-ray irradiator 20, an X-ray detector 26, and an arm 30. The couch unit 10 includes a table 11 on which a subject P is laid, and a table supporting base 12 that supports the table 11 in such a manner that the table 11 is movable in, for example, the longitudinal and vertical directions thereof. The X-ray irradiator 20 emits X-rays to the subject P that is laid in the couch unit 10. The X-ray detector 26 detects X-rays emitted by the X-ray irradiator 20 and having passed through the subject P. The arm 30 movably holds the X-ray irradiator 20 and the X-ray detector 26.

The X-ray diagnosis apparatus 100 further includes a moving mechanism unit 31 that drives the couch unit 10 and the arm 30, an image processor 40 that generates image data on the basis of a detection signal detected by the X-ray detector 26, an image data collector 50 that collects and saves the image data generated by the image processor 40, a display unit 60 that displays the image data generated by the image processor 40, first and second operation units 71 and 72 for inputting information such as various commands, and a system controller 90 that controls the above-described units.

The X-ray irradiator 20 includes an X-ray generator 21 that emits X-rays to the subject P, and a high voltage generator 22 that generates a high voltage necessary for the X-ray generator 21 to emit X-rays. Furthermore, the X-rav generator 21 includes an X-ray tube 23 that generates X-rays to be emitted to the subject P, an X-ray collimator device 24 that allows an emission port, through which X-rays emitted from the X-ray tube 23 pass, to be changeable, and an X-ray collimator controller 25 that controls the X-ray collimator device 24.

The X-ray collimator device 24 includes a plurality of collimator blades and a blade moving mechanism that moves these collimator blades, and forms an emission aperture having, for example, a rectangular shape through which X-rays emitted from the X-ray tube 23 pass. Thus, the X-ray collimator device 24 allows the emission aperture to be changeable, and restricts the range of emission of X-rays to the subject P. Based on the distance between the X-ray tube 23 and an X-ray detecting device 27, the distance between the X-ray tube 23 and the X-ray collimator device 24, and the dimension of a detection surface of the X-rav detecting device 27 that detects X-rays, which are all provided by the system controller 90, the X-ray collimator controller 25 moves the respective blades so that a range into which X-rays are emitted from the X-ray tube 23 can be included in the detection surface of the X-ray detecting device 27.

The high voltage generator 22 includes a high voltage generating device that supplies a high voltage to the X-ray tube 23 of the X-ray generator 21, and an X-ray controller that controls this high voltage generator. Thus, the high voltage generator 22 supplies to the X-ray tube 23 a high voltage based on a tube voltage and a tube current for fluoroscopy, a tube voltage and a tube current for photography, and X-ray emission conditions such as an emission period, which are all provided by the system controller 90. The high voltage is used in generation of X-rays for fluoroscopy or X-rays for photography, which are X-rays that give a higher dose than X-rays for fluoroscopy.

The X-ray detector 26 includes the X-ray detecting device 27 that detects X-rays having been emitted through the emission aperture of the X-ray collimator device 24 in the X-ray irradiator 20 and having passed through the subject P, and a signal processor 28 that generates X-ray projection data by processing a detection signal detected by the X-ray detecting device 27. The X-ray detector 26 then outputs the generated X-ray projection data to the image processor 40. The arm 30 holds the X-ray generator 21 of the X-ray irradiator 20 at one end thereof, and holds the X-ray detector 26 at the other end thereof.

The moving mechanism unit 31 includes a table moving mechanism 32 that moves the table 11 of the couch unit 10 in, for example, the longitudinal and vertical directions of the table 11, and an arm moving mechanism 33 that moves the X-ray generator 21 of the X-ray irradiator 20 and the X-ray detector 26. Thus, the arm moving mechanism 33 drives the arm 30 so as to slide it, thereby rotating the X-ray generator 21 and the X-ray detector 26 in a direction in which the arm 30 is slid. Additionally, the arm moving mechanism 33 drives the arm 30 so as to rotate it, thereby rotating the X-ray generator 21 and the X-ray detector 26 in a direction perpendicular to the direction in which the arm 30 is slid.

The image processor 40 includes an image data generator 41 that generates image data based on the X-ray projection data generated by the signal processor 28 of the X-ray detector 26 as a result of emission of X-rays to the subject P. The image processor 40 also includes a spot-area setting unit 42 that sets a spot area of the image data generated by the image data generator 41. The image processor 40 further includes an image data processor 43 that performs, on the basis of the spot area set by the spot-area setting unit 42, composition of the image data generated by the image data generator 41.

When input for starting fluoroscopy is fed by the first operation unit 71 or the second operation unit 72, the image data generator 41 generates image data having a predetermined frame rate, from the X-ray projection data generated by the X-ray detector 26 as a result of X-ray emission for fluoroscopy. The image data generator 41 then outputs the generated image data frame by frame to the image data processor 43. Furthermore, when input for setting a spot area is fed after input for starting fluoroscopy is fed, the image data generator 41 outputs the image data frame by frame to the spot-area setting unit 42 and the image data processor 43.

For example, while the X-ray irradiator 20 is emitting X-rays to a given range of the subject P with a device such as a catheter for intervention or a wire for treatment being inserted into the given range, the spot-area setting unit 42 outputs positional data indicating the position of the spot area to the image data processor 43 and to the system controller 90 in response to input fed by the first operation unit 71 for setting an area corresponding to a region of interest in image data formed of a plurality of frames generated by the image data generator 41 in a time sequence.

Thereafter, the X-ray collimator controller 25 of the X-ray irradiator 20 causes the X-ray collimator device 24 to narrow the emission aperture based on the positional data of the spot area supplied from the system controller 90, so that X-rays can be emitted to a range corresponding to the spot area, which is smaller than the given range.

The image data processor 43 stores, in its internal memory, the positional data of the spot area when input for setting the spot area is fed, and image data of, for example, a single frame generated by the image data generator 41. The image data processor 43 then composites still image data obtained by excluding data of the spot area from the image data stored in the internal memory, with image data generated by emitting X-rays to the range corresponding to the spot area. The image data processor 43 then outputs thus-obtained composite image data to the display unit 60.

The display unit 60 includes a monitor constructed of a liquid crystal panel or a cathode-ray tube (CRT), and displays image data generated by the image data generator 41 of the image processor 40. The display unit 60 also displays composite image data obtained by the image data processor 43 of the image processor 40.

The first operation unit 71 is an interactive interface to be operated by the hands of the operator and is provided with an input device, such as a keyboard, a trackball, a joystick, or a mouse, and a display panel and further provided with, for example, various switches. The first operation unit 71 feeds, for example, input for setting a condition for X-ray emission, input for setting a spot area, emission-starting input for emitting X-rays, emission-stopping input for stopping X-ray emission, and input for setting information associated with operational information that is input from the second operation unit 72.

The second operation unit 72 is placed on the floor on which the couch unit 10 is installed, and includes a plurality of input units to be operated by a foot of the operator. The second operation unit 72 feeds, for example, emission-starting input for emitting X-rays, emission-stopping input for stopping X-ray emission, input for collecting image data, input for moving the table 11 of the couch unit 10, and input for moving the X-ray generator 21 and the X-ray detector 26 on the basis of the information associated with operational information that is input from the first operation unit 71.

The system controller 90 includes a central processing unit (CPU) and a storage circuit. The system controller 90 temporarily stores pieces of input information input from the first operation unit 71 and the second operation unit 72, and then collectively controls the X-ray irradiator 20, the X-ray detector 26, the moving mechanism unit 31, the image processor 40, and the image data collector 50 on the basis of these pieces of input information.

The following describes in detail the configuration of and operations for the second operation unit 72 with reference to FIG. 1 to FIG. 3.

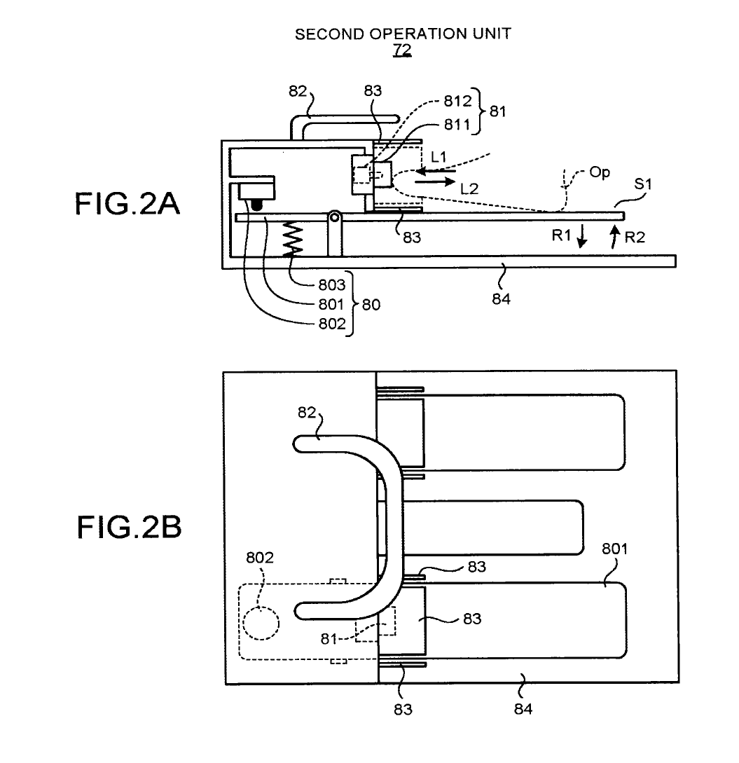

FIG. 2A and FIG. 2B are views illustrating one example of the configuration of the second operation unit 72. FIG. 2A is a side view of the second operation unit 72, and FIG. 2B is a top view of the second operation unit 72.

This second operation unit 72 includes input units including: at least one set of input units including a first input unit 80 for inputting first operational information, a second input unit 81 for inputting second operational information, and a third input unit 82 for inputting initialization information. The second operation unit 72 further includes four guides 83 that guide the foot of the operator to the second input unit 81, and a supporting body 84 that supports the respective input units and the guides 83.

The first input unit 80 is arranged so as to be movable by being pedaled, and is used for inputting the first operational information in response to a pedaling operation performed by the operator. Specifically, the first input unit 80 includes a first operative unit 801, a first switch 802, and a spring 803. The first operative unit 801 is arranged apart from the second input unit 81 and arranged so as to be moved by being pedaled by an operating body Op, that is, a foot of the operator, and has the upper face formed in a rectangular shape. The first switch 802 is turned on in response to a pedaling operation performed on the first operative unit 801. The spring 803 that expands and contracts so that the first operative unit 801 is maintained at a stop position S1.

The first operative unit 801 is arranged in such a manner that: the upper surface thereof, which is arranged so as to be horizontal when the first operative unit 801 is at the stop position S1, is positioned below the second input unit 81; and the first operative unit 801 can be tilted in a direction indicated by the arrow R1, which is downward from the stop position S1, with the axis of tilting serving as the supporting point. The axis of tilting is supported by a part of the supporting body 84 that is provided between one end and the central part of the first operative unit 801 in the longitudinal direction thereof. The first switch 802 is arranged above the one end of the first operative unit 801. The spring 803 is arranged so as to be expandable with tilting of the first operative unit 801 in the direction of R1.

When a pedaling operation is performed with the operating body Op, the first operative unit 801 having been at the stop position S1 tilts in the direction of R1 and its upper face on the one end is pressed down, which turns on the first switch 802. When an operation to release the first operative unit 801 is performed in which the operating body Op is detached from the first operative unit 801, the first switch 802 is turned off with the first operative unit 801 released by tilting in a direction indicated by the arrow R2, i.e., a direction opposite to the direction of R1 due to the repelling force of the spring 803.

The second input unit 81 is arranged apart from the first input unit 80 and positioned so that the operator can touch the second input unit 81 while touching the upper surface of the first input unit 80, which faces away from the floor surface of a room. The second input unit 81 is used for inputting the second operational information in response to an operation performed by the operator. Specifically, the second input unit 81 includes: a second operative unit 811 positioned so that it can be pressed and so that the operating body Op can touch it while touching the upper surface of the first operative unit 801 of the first input unit 80; and a second switch 812 that is turned on by a pressing operation on the second operative unit 811.

The second operative unit 811 is positioned above a part of the first operative unit 801 between the axis of tilting and the central part thereof in the longitudinal direction thereof, has the central part of the outer circumference supported by the supporting body 84, and has a columnar part arranged so as to be movable in a direction indicated by the arrow L1, which is the horizontal direction. The second switch 812 is arranged inside the second operative unit 811.

Thus, with a pressing operation performed on the second operative unit 811 with the operating body Op moving in the direction of L1 following the longitudinal direction of the first operative unit 801, the part of the second operative unit 811 moves in the direction of L1 to turn on the second switch 812. More specifically, the second input unit 81 is provided so that it can be pressed in the longitudinal direction of the first input unit 80. Furthermore, in response to an operation of releasing the second operative unit 811 with the operating body Op separated from the second operative unit 811, the part of the second operating unit 811 moves in a direction indicated by the arrow L2, which is a direction opposite to the direction of L1, to turn off the second switch 812.

The third input unit 82 is arranged on an upper part of the supporting body 84 so that the third input unit 82 can be pressed down, and the initialization information is input in response to a pressing operation performed on the third input unit 82.

The guide 83 has parts arranged above and beneath the second operative unit 811 so as to be able to become parallel to the upper surface of the first operative unit 801 when the first operative unit 801 is at the stop position S1. The guide 83 also has parts arranged to the opposite sides of the second operative unit 811 so that the parts may be parallel to each other.

The following describes one example of controlling operation of the system controller 90 to control, for example, X-ray emission from the X-ray irradiator 20 on the basis of the first and the second operational information input from the second operation unit 72.

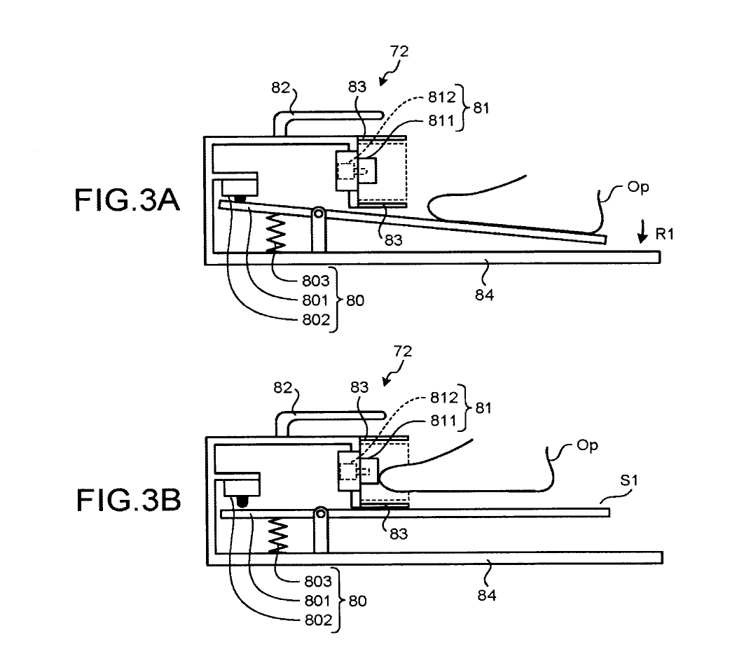

FIG. 3A and FIG. 3B are views illustrating examples of operations performed on the second operation unit 72. FIG. 3A illustrates a pedaling operation for inputting the first operational information. FIG. 3B illustrates a pressing operation for inputting the second operational information.

The first input unit 80 of the second operation unit 72 receives the first operational information when the first switch 802 is turned on, as illustrated in FIG. 3A, in response to a pedaling operation performed on the first operative unit 801 by the operating body Op touching the upper face of the first operative unit 801 having been stopped at the stop position S1. Input of the first operational information is stopped when the first switch 802 is turned off in response to a releasing operation performed on the first operative unit 801 with the operating body Op.

The second input unit 81 receives the second operational information when the second switch 812 is turned on, as illustrated in FIG. 3B, in response to the pressing operation performed on the second operative unit 811 by the operating body Op moving in the direction of L1 following the longitudinal direction of the first operative unit 801. Input of the second operational information is stopped when the second switch 812 is turned off in response to a releasing operation performed on the second operative unit 811.

The second operative unit 811 is thus positioned so that the operating body Op can touch the second operative unit 811 while touching the upper surface of the first operative unit 801. As a result of this positioning, a small movement of the operating body Op from one unit of the first operative unit 801 and the second operative unit 811 is sufficient to enable or stop input of operational information corresponding to operation of the other unit.

The system controller 90 stores: information on first and second modes set in association with the first and the second operational information according to input from the first operation unit 71; and switching information for causing operation to switch from one mode to the other mode of the first and the second modes. The system controller 90 then controls the high voltage generator 22 and the X-ray collimator controller 25 of the X-ray irradiator 20, the X-ray detector 26, the moving mechanism unit 31, the image processor 40, and the image data collector 50 on the basis of: the information on the first and the second modes; the switching information; and the first operational information, the second operational information, and the initialization information input by the first to the third input units 80, 81, and 82, respectively. For example, the system controller 90 performs control for changing the first and the second operational information or either thereof on the basis of the operational information input from both or either of the first input unit 80 and the second input unit 81.

In response to input of the initialization information, the system controller 90 instructs the image processor 40 to cause the display unit 60 to display the information on the first mode. In response to the n-th input (n is an odd positive integer) of the second operational information after input of the initialization information, the system controller 90 causes the display unit 60 to display the information on the second mode. Additionally, in response to the (n+1)-th input of the second operational information, the system controller 90 causes the display unit 60 to display the information on the first mode. That is, the display unit 60 performs display for indicating the first operational information, thereby displaying the function that is currently assigned.

After input of the initialization information, the system controller 90 activates operation corresponding to the first mode in response to input of the first operational information before the second operational information is input. The system controller 90 causes operation to switch from the first mode to the second mode in response to the n-th input of the second operational information after input of the initialization information, and activates operation corresponding to the second mode in response to input of the first operational information after the n-th input of the second operational information. Furthermore, the system controller 90 causes operation to switch from the second mode to the first mode in response to the (n+1)-th input of the second operational information, and activates operation corresponding to the first mode in response to input of the first operational information after the (n+1)-th input of the second operational information.

Here, when fluoroscopy and photography modes, for which X-ray emission from the X-ray irradiator 20 is controlled, have been set as the first and the second modes, the system controller 90 performs initialization in response to input of the initialization information so that the X-ray irradiator 20 can emit X-rays for fluoroscopy. The system controller 90 causes the X-ray irradiator 20 to emit X-rays for fluoroscopy in response to input of the first operational information before input of the second operational information, causes the X-ray irradiator 20 to emit X-rays for photography in response to input of the first operational information after the n-th input of the second operational information, and causes the X-ray irradiator 20 to emit X-rays for fluoroscopy in response to input of the first operational information after the (n+1)-th input of the second operational information. That is, the system controller 90 performs control by causing X-ray emission from the X-ray irradiator 20 to switch between the fluoroscopy and the photography purposes, on the basis of the second operational information.

Thus, when the operating body Op is performing a pedaling operation to activate X-ray emission for one of the fluoroscopy and the photography purposes, a pressing operation for causing X-ray emission to switch to the other purpose can be performed easily and quickly with a small movement of the operating body Op from the first operative unit 801. When the operating body Op is performing a pressing operation to cause X-ray emission to switch to the other purpose, a pedaling operation for activating X-ray emission for the other purpose can be performed easily and quickly with a small movement of the operating body Op from the second operative unit 811. This embodiment eliminates the need to provide two separate first operative units 801 for fluoroscopy and photography on each of which a pedaling operation can be performed, thereby preventing errors in operation due to errors in pedaling.

Alternatively, when fluoroscopy and spot fluoroscopy modes, for which X-ray emission from the X-ray irradiator 20 is controlled, have been set as the first and the second modes, the system controller 90 performs initialization in response to input of the initialization information from the third input unit 82 so that the X-ray irradiator 20 can emit X-rays for fluoroscopy to the given range. The system controller 90 then, in response to input of the first operational information before input of the second operational information, causes the X-ray irradiator 20 to emit X-rays for fluoroscopy to the given range. The system controller 90 then, in response to input of the first operational information after the n-th input of the second operational information, causes the X-ray irradiator 20 to emit X-rays for fluoroscopy to the range corresponding to the predetermined spot area, which is smaller than the given range. Furthermore, in response to input of the first operational information after the (n+1)-th input of the second operational information, the system controller 90 causes the X-ray irradiator 20 to emit X-rays for fluoroscopy to the given range.

Thus, when the operating body Op is performing a pedaling operation to emit X-rays for fluoroscopy to one range of the given range and the range corresponding to the spot area, a pressing operation for causing X-ray emission for fluoroscopy to switch to the other range can be performed easily and quickly with a small movement of the operating body Op from the first operative unit 801. Additionally, when the operating body Op is performing the pressing operation to cause X-ray emission for fluoroscopy to switch to the other range, a pedaling operation for emitting X-rays for fluoroscopy to the other range can be performed easily and quickly with a small movement of the operating body Op from the second operative unit 811.

Alternatively, when fluoroscopy and fluoroscopy collection modes, for which X-ray emission from the X-ray irradiator 20 and image data collection are controlled, have been set as the first mode and the second mode, the system controller 90 performs initialization in response to input of the initialization information from the third input unit 82 so that the X-ray irradiator 20 can emit X-rays for fluoroscopy. In response to input of the first operational information before input of the second operational information, the system controller 90 causes the X-ray irradiator 20 to emit X-rays for fluoroscopy. In response to input of the first operational information after the n-th input of the second operational information, the system controller 90 causes the X-ray irradiator 20 to emit X-rays for fluoroscopy and causes the image data collector 50 to collect and store image data generated with X-rays for fluoroscopy. In response to input of the first operational information after the (n+1)-th input of the second operational information, the system controller 90 causes the X-ray irradiator 20 to emit X-rays for fluoroscopy.

As described above, when the operating body Op is performing a pedaling operation to activate operation corresponding to one mode of the first and the second modes, a pressing operation for causing operation to switch to the other mode can be performed on the second operative unit 811 with a small movement of the operating body Op from the first operative unit 801. Additionally, when the operating body Op is performing a pressing operation to cause operation to switch to the other mode, a pedaling operation for activating operation corresponding to the other mode can be performed on the first operative unit 801 with a small movement of the operating body Op from the second operative unit 811. Thus, the pressing operation for causing operation to switch to the other mode and the pedaling operation for activating operation corresponding to the other mode can be performed easily and quickly while kicking off the second operation unit 72 and committing errors in operation are prevented.

Further alternatively, when fluoroscopy and temporary-stop modes, for which X-ray emission from the X-ray irradiator 20 is controlled, have been set as the first mode and the second mode, the system controller 90 causes the X-ray irradiator 20 to emit X-rays for fluoroscopy in response to input of the first operational information before input of the second operational information after input of the initialization information from the third input unit 82, then stops the X-ray irradiator 20 from emitting X-rays in response to the first input of the first operational information after input of the second operational information, and causes the X-ray irradiator 20 to emit X-rays for fluoroscopy in response to the second input of the first operational information after the first input of the first operational information is stopped.

As described above, when the operating body Op is performing a pedaling operation to emit X-rays for fluoroscopy, a pressing operation for stopping the X-ray emission can be performed on the second operative unit 811 easily and quickly with a small movement of the operating body Op from the first operative unit 801. Additionally, when the operating body Op is performing a pressing operation on the second operative unit 811 to stop the X-ray emission, a pedaling operation for emitting X-rays for fluoroscopy can be performed quickly and easily on the first operative unit 801 with a small movement of the operating body Op from the first operative unit 801.

This embodiment may be carried out by employing, instead of a single-plane type formed of the X-ray irradiator 20 and the X-ray detector 26 as described above, a biplane type formed of two first and second X-ray irradiators that emit X-rays to the subject P and first and second X-ray detectors that detect X-rays emitted from the first and the second X-ray irradiators. In this case, if information of first to third emission modes is set as first to third modes, the system controller 90 causes the first X-ray irradiator to emit X-rays in response to input of first operational information before second operational information, causes the second X-ray irradiator to emit X-rays in response to input of the first operational information after the (3m−2)-th input (m is a positive integer) of the second operational information, causes the first and the second X-ray irradiators to emit X-rays in response to input of the first operational information after the (3m−1)-th input of the second operational information, and causes the first irradiator to emit X-rays in response to input of the first operational information after the 3m-th input of the second operational information.

As described above, when the operating body Op is performing a pedaling operation, each of the following operations can be performed easily and quickly with a small movement thereof from the first operative unit 801: a pressing operation for causing X-ray emission to switch from the first X-ray irradiator to the second X-ray irradiator; a pressing operation for causing X-ray emission to switch from the second X-ray irradiator to the first and the second X-ray irradiators; a pressing operation for causing X-ray emission to switch from the first and the second X-ray irradiators to the first X-ray irradiator; and a pedaling operation for activating X-ray emission after such switching.

According to the first embodiment described above, the second operative unit 811 is positioned so that the operating body Op can touch the second operative unit 811 while touching the upper surface of the first operative unit 801. As a result of this positioning, when the operating body Op is performing a pedaling operation to activate operation corresponding to one mode of the first and the second modes, a pressing operation for causing operation to switch to the other mode can be performed on the second operative unit 811 with a small movement of the operating body Op from the first operative unit 801. Additionally, when the operating body Op is performing a pressing operation to cause operation to switch to the other mode, a pedaling operation for activating operation corresponding to the other mode can be performed on the first operative unit 801 with a small movement of the operating body Op from the second operative unit 811.

Thus, the pressing operation for causing operation to switch to the other mode and the pedaling operation for activating operation corresponding to the other mode can be easily and quickly performed while kicking off the second operation unit 72 and committing errors in operation are prevented.

### Second Embodiment

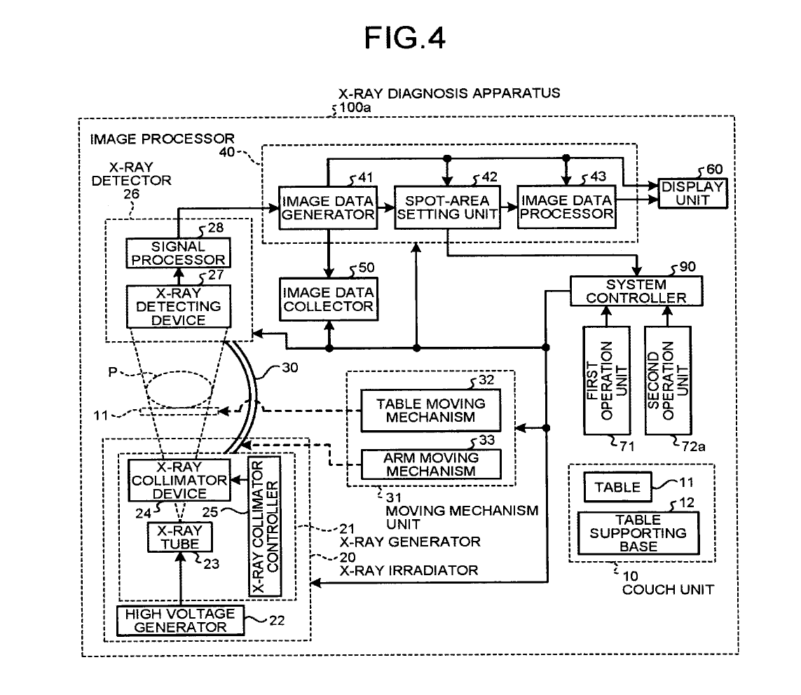

FIG. 4 is a block diagram illustrating the configuration of an X-ray diagnosis apparatus according to a second embodiment. This X-ray diagnosis apparatus 100a differs from the X-ray diagnosis apparatus 100 according to the first embodiment illustrated in FIG. 1 in that it includes a second operation unit 72a in place of the second operation unit 72 of the X-ray diagnosis apparatus 100. Units included in the X-ray diagnosis apparatus 100a that have the same configurations and the same functions as those of the X-ray diagnosis apparatus 100 illustrated in FIG. 1 are assigned the same reference signs, and detailed descriptions thereof are omitted.

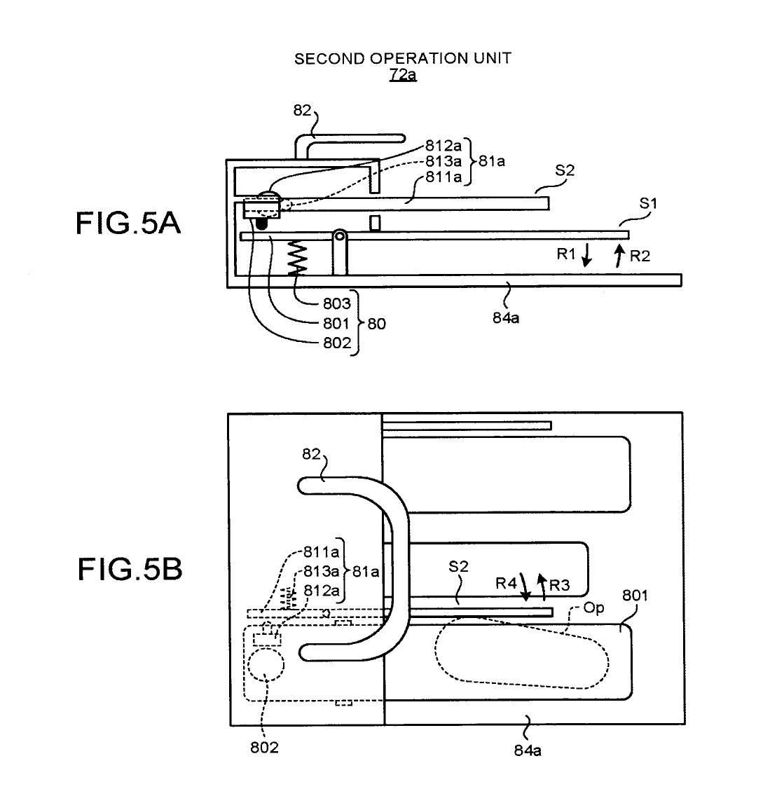

FIG. 5A and FIG. 5B are views illustrating the configuration of the second operation unit 72a. FIG. 5A is a side view of the second operation unit 72a. FIG. 5B is a top view of the second operation unit 72a.

This second operation unit 72a includes input units and a supporting body 84a supporting the respective input units. The input units include: at least one set of input units, including the first input unit 80 for inputting first operational information, a second input unit 81a for inputting second operational information, and the third input unit 82 for inputting initialization information. Units included in the second operation unit 72a that have the same configurations and the same functions as those of the second operation unit 72 according to the first embodiment illustrated in FIGS. 2A and 2B are assigned the same reference signs, and detailed descriptions thereof are omitted.

The second input unit. 81a is spaced from the first input unit 80, and includes a second operative unit 811a, a second switch 812a, and a spring 813a. The second operative unit 811a is positioned so that the operating body Op can touch the second operative unit 811a while touching the upper surface of the first operative unit 801 of the first input unit 80, so as to be able to tilt. The second switch 812a is turned on by a tilting operation performed on the second operative unit 811a. The spring 813a expands and contracts so as to be able to maintain the second operative unit 811a at a stop position S2.

The second operative unit 811a is arranged so as to be positioned above, but not vertically overlapping, the upper surface of the first operative unit 801 when at the stop position S2, and so that the longitudinal direction thereof can be parallel to the longitudinal direction of the first operative unit 801. The second operative unit 811a is arranged so as to be able to tilt in a direction other than the vertical direction with the axis of tilting serving as the supporting point. The direction other than the vertical direction is, for example, a direction indicated by the arrow R3, which is the horizontal direction. The axis of tilting is supported by a part of the supporting body 84a, and this part is provided between one end and the center of the second operative unit 811a in the longitudinal direction thereof. In other words, the second input unit 81a is provided so that it can be pressed by horizontally changing the angle of a foot of the operator that is placed on the upper surface of the first input unit 80.

The second switch 812a is arranged near one end of the second operative unit 811a. The spring 813a is arranged so as to be able to expand when the second operative unit 811a tilts in the direction of R3. The second switch 612a is turned on when the second operative unit 811a tilts from the stop position S2 in the direction of R3. The second switch 812a is turned off when the second operative unit 811a after having tilted in the direction of R3 tilts in a direction indicated by the arrow R4, which is a direction opposite to the direction of R3, due to the repelling force of the spring 813a.

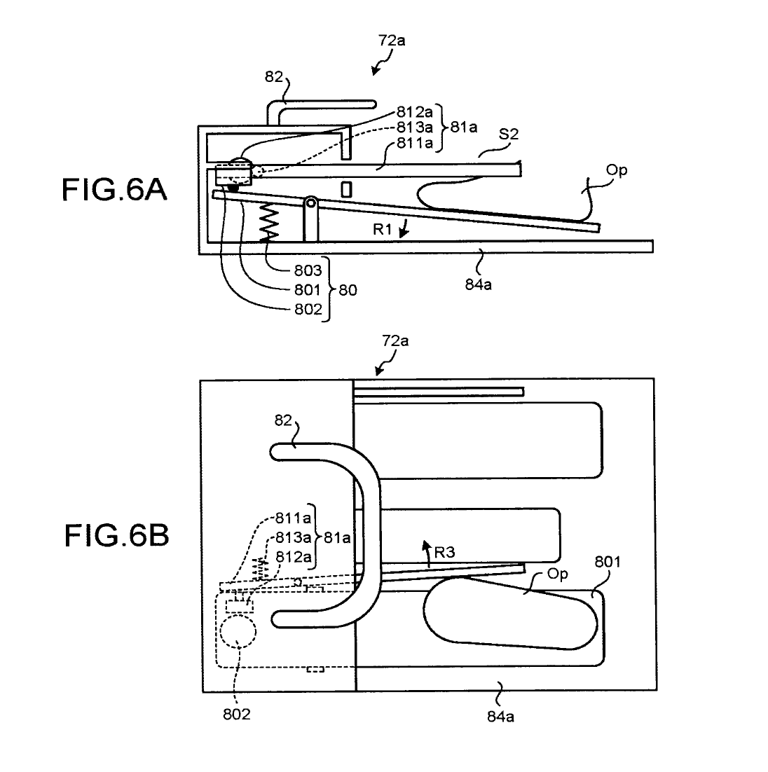

Furthermore, as illustrated FIG. 6A, the second input unit. 81a receives the second operational information when the second switch 812a is turned on in response to a tilting operation on the second operative unit 811a by the operating body Op that moves in a direction other than the longitudinal direction of the first operative unit 801. Input of the second operational information is stopped when the second switch 812a is turned off in response to a releasing operation on the second operative unit 811a by the operating body Op.

As described above, the second operative unit 811a is positioned so that the operating body Op can touch the second operative unit 811a while touching the upper surface of the first operative unit 801. As a result of this positioning, a small movement of the operating body Op from one of the first operative unit 801 and the second operative unit 811a is sufficient to enable input of the operational information corresponding to operation of the other unit.

The system controller 90 stores: information on the first and the second modes, such as the fluoroscopy and the photography modes, the fluoroscopy and the temporary-stop modes, the fluoroscopy and the spot fluoroscopy modes, or the fluoroscopy and the fluoroscopy collection modes, which is set in association with the first and the second operational information in accordance with input from the first operation unit 71; and switching information for causing operation to switch from one mode to the other mode of the first and the second modes, which is set in association with the second operational information. The system controller 90 controls the X-ray irradiator 20, the X-ray detector 26, the moving mechanism unit 31, the image processor 40, and the image data collector 50 on the basis of the information on the first and the second modes, the switching information, the first operational information, the second operational information, and initialization information input from the first to the third input units 80, 81a, and 82, respectively. In the same manner as in the first embodiment, the system controller 90 then, in response to input of the first operational information and the second operational information, causes operation corresponding to the first mode and operation corresponding to the second mode to be executed.

Here, when the first and the second modes, such as the fluoroscopy and the photography modes, the fluoroscopy and the spot fluoroscopy modes, or the fluoroscopy and the fluoroscopy collection modes, have been set, the system controller 90 causes the display unit 60 to: display information on the first mode in response to input of the initialization information; display information on the second mode in response to the n-th input (n is an odd positive integer) of the second operational information after input of the initialization information; and display information on the first mode in response to the (n+1)-th input of the second operational information. The system controller 90 further causes, in response to input of the first operational information before input of the second operational information after input of the initialization information, operation corresponding to the first mode to be executed, causes, in response to input of the first operational information after the n-th input of the second operational information, operation corresponding to the second mode, and causes, in response to input of the first operational information after the (n+1)-th input of the second operational information, operation corresponding to the first mode to be executed.

As described above, when the operating body Op is performing a pedaling operation to activate operation corresponding to one of the first and the second modes, a small movement thereof from the first operative unit 801 is sufficient to enable a tilting operation on the second operative unit 811a for causing operation to switch to the other mode. Additionally, when the operating body Op is performing the tilting operation for causing operation to switch to the other mode, a small movement thereof from the second operative unit 811a is sufficient to enable a pedaling operation on the first operative unit 801 for activating operation corresponding to the other mode. Thus, the tilting operation for causing operation to switch to the other mode and the pedaling operation for activating operation corresponding to the other mode can be performed easily and quickly while kicking off the second operation unit 72a and committing errors in operation are prevented.

Here, when the fluoroscopy and the temporary-stop modes are set as the first and the second modes, the system controller 90 activates X-ray emission for fluoroscopy in response to input of the first operational information before input of the second operational information, stops X-ray emission in response to the 1st input of the first operational information after input of the second operational information, and activates X-ray emission for fluoroscopy in response to the 2nd input of the first operational information after the 1st input of the first operational information is stopped.

Alternatively, when information on first and second positions of the table 11 is set as the first and the second modes, the system controller 90 moves the table 11 to the first position in response to input of the first operational information before input of the second operational information, moves the table 11 to the second position in response to input of the first operational information after the n-th input of the second operational information, and moves the table 11 to the first position in response to input of the first operational information after the (n+1)-th input of the second operational information.

Furthermore, when employing, instead of a single-plane type formed of the X-ray irradiator 20 and the X-ray detector 26 as described above, a biplane type formed of first and second X-ray irradiators and first and second X-ray detectors that detect X-rays emitted from the first and the second X-ray irradiators, the embodiment may be carried out as follows. The system controller 90 causes the first X-ray irradiator to emit X-rays in response to input of the first operational information before input of the second operational information, causes the second X-ray irradiator to emit X-rays in response to input of the first operational information after the (3m−2)-th input (m is a positive integer) of the second operational information, causes the first and the second X-ray irradiators to emit X-rays in response to input of the first operational information after the (3m−1)-th input of the second operational information, and causes the first irradiator to emit X-rays in response to input of the first operational information after the 3m-th input of the second operational information.

According to the second embodiment described above, the second operative unit 811a is positioned so that the operating body Op can touch the second operative unit 811a while touching the upper surface of the first operative unit 801. With this positioning, when the operating body Op is performing a pedaling operation to activate operation corresponding to one mode of the first and the second modes, a small movement thereof from the first operative unit 801 is sufficient to enable a tilting operation on the second operative unit 811a for causing operation to switch to the other mode. Additionally, when the operating body Op is performing a tilting operation to cause operation to switch to the other mode, a small movement thereof from the second operative unit 811a is sufficient to enable a pedaling operation on the first operative unit 801 for activating operation corresponding to the other mode.

Thus, a tilting operation for causing operation to switch to the other mode and a pedaling operation for activating operation corresponding to the other mode can be easily and quickly performed while kicking off the second operation unit 72a and committing errors in operation are prevented.

The above second embodiment illustrates a case where the second input unit 81a is provided nearer the middle than the side of the second operation unit 72a. However, the embodiment is not limited thereto, and the second input unit 81a may be provided at any desired position. For example, the second input unit 81a may be provided nearer the side than the middle of the second operation unit 72a. Such a case is described here with reference to FIG. 5A and FIG. 5B. The second input unit 81a is provided at two positions facing each other with the operating body Op therebetween, and the second switch 812a is tuned on when the second operative unit 811a tilts in the direction indicated by the arrow R4. The second switch 812a is turned off when, after having tilted in the direction of R4, the second operative unit 811a tilts in the direction indicated by the arrow R3, which is a direction opposite to the direction of R4, due to the repelling force of the spring 813a.

Here, the second input unit 81a not only may be provided along a lateral side of the operating body Op, as described above, but also may be provided above the operating body Op. For example, the second operation unit 72a may have a boxlike shape into which the operating body Op can be inserted, such that the second input unit 81a is placed on the upper surface of the operating body Op when the operating body Op is inserted into the box. This example is described here with reference to FIG. 5A and FIG. 5B. The second input unit 81a is provided along one side of the operating body Op that is opposite to the side thereof closer to the first input unit 80, and the second switch 812a is turned on when the second operative unit 811a tilts in a direction indicated by the arrow R2. The second switch 812a is turned off when the second operative unit 811a having tilted in the direction of R2 tilts, due to the repelling force of the spring 813a, in a direction indicated by the arrow R1, which is a direction opposite to the direction of R2.

The second input unit 81a may be thus provided at any desired position. Furthermore, not only the second input unit 81a may be provided at any one of the above-described positions, but also the second input units 81a may be provided at a plurality of positions. In the latter case, operational information input by the second input units 81a at the respective positions may be different kinds of information.

### Third Embodiment

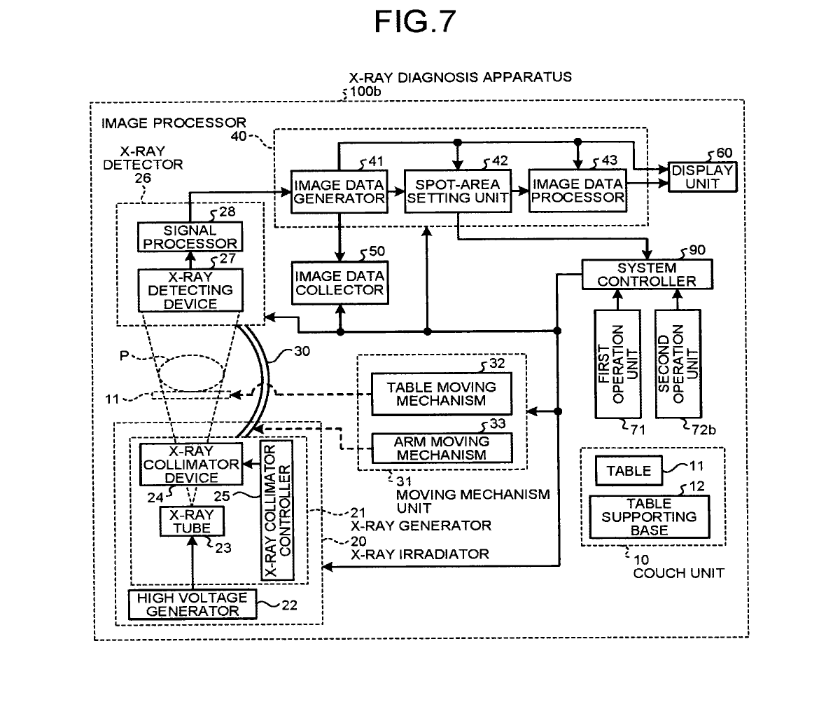

FIG. 7 is a block diagram illustrating the configuration of an X-ray diagnosis apparatus according to a third embodiment. This X-ray diagnosis apparatus 100b differs from the X-ray diagnosis apparatus 100 according to the first embodiment illustrated in FIG. 1 in that it includes a second operation unit 72b in place of the second operation unit 72 of the X-ray diagnosis apparatus 100. Units included in the X-ray diagnosis apparatus 100b that have the same configurations and the same functions as those of the X-ray diagnosis apparatus 100 illustrated in FIG. 1 are assigned the same reference signs, and detailed descriptions thereof are omitted.

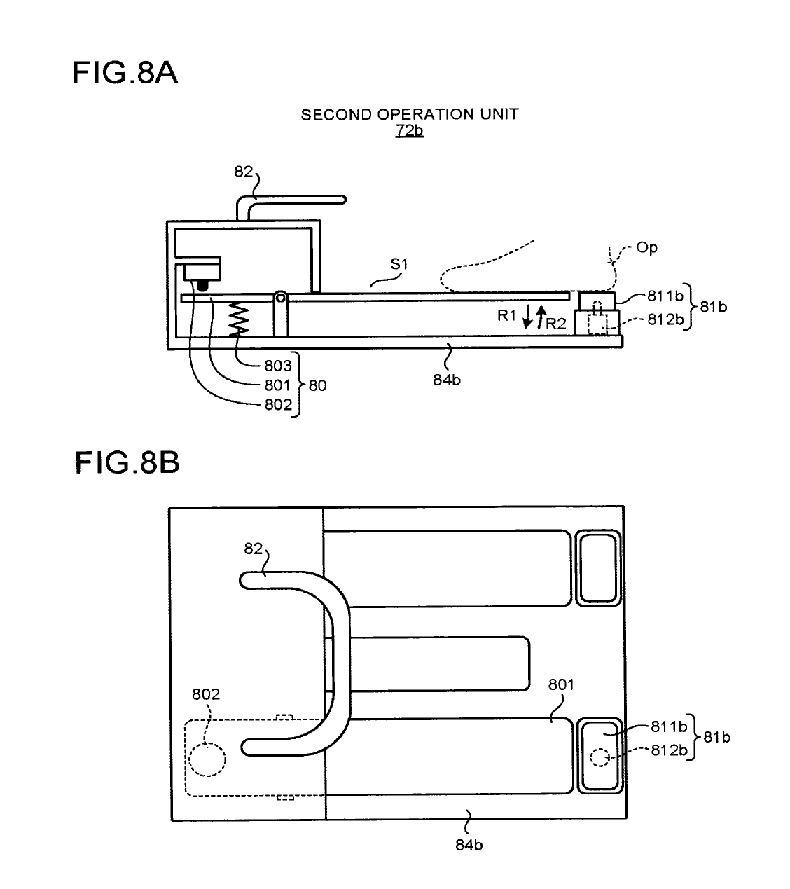

FIG. 8A and FIG. 8B are diagrams illustrating the configuration of the second operation unit 72b. FIG. 8A is a side view of the second operation unit 72b. FIG. 8B is a top view of the second operation unit 72b.

This second operation unit 72b differs from the second operation unit 72 according to the first embodiment illustrated in FIGS. 2A and 2B in that it includes a second input unit arranged near one end of the first operative unit 801 of the first input unit 80 in the longitudinal direction thereof. Units included in the second operation unit 72b that have the same configurations and the same functions as those of the second operation unit 72 according to the first embodiment illustrated in FIGS. 2A and 2B are assigned the same reference signs, and detailed descriptions thereof are omitted.

The second operation unit 72b includes input units including: at least one set of input units including the first input unit 80 for inputting first operational information, a second input unit 81b for inputting second operational information, and the third input unit 82 for inputting initialization information. The second operation unit 72b further includes a supporting body 84b supporting the respective input units.

The second input unit 81b is arranged apart from the first input unit 80, and includes a second operative unit 811b and a second switch 812b. The second operative unit 811b is positioned so that it can be pressed and so that the operating body Op can touch it while touching the upper surface of the first operative unit 801. The second switch 812b is turned on by a pressing operation on the second operative unit 811b.

The second operative unit 811b is positioned near one end of the first operative unit 801 in the longitudinal direction thereof, has the bottom thereof supported by the supporting body 84b, and includes a columnar upper section arranged so as to be movable downward. When the second switch 812b is turned on in response to a pressing operation on the second operative unit 811b by the operating body Op, the second operational information is input. When the second switch 812b is turned off in response to a releasing operation on the second operative unit 811b, input of the second operational information is stopped.

The second operative unit 811b is thus positioned so that the operating body Op can touch the second operative unit 811b while touching the upper surface of the first operative unit 801. With this positioning, a small movement of the operating body Op from one of the first operative unit 801 and the second operative unit 811b is sufficient to enable input of operational information corresponding to operation of the other.

The system controller 90 stores: information on the first and the second modes, such as fluoroscopy and photography modes, fluoroscopy and spot fluoroscopy modes, or fluoroscopy and fluoroscopy collection modes set in association with the first and the second operational information based on input from the first operation unit 71; and switching information for causing operation to switch from one mode to the other of the first and the second modes. The system controller 90 then controls the X-ray irradiator 20, the X-ray detector 26, the moving mechanism unit 31, the image processor 40, and the image data collector 50 on the basis of the information on the first and the second modes, the switching information, the first operational information, the second operational information, and the initialization information. The system controller 90 thus activates operation corresponding to the first mode or operation corresponding to the second mode in response to input of the first operational information or the second operational information, in the similar manner as in the first embodiment.

Here, when the fluoroscopy and the photography modes, the fluoroscopy and the spot fluoroscopy modes, the fluoroscopy and the fluoroscopy collection modes or the like have been set as the first and the second modes, the system controller 90 activates operation corresponding to the first mode in response to input of the first operational information before input of the second operational information after input of the initialization information, activates operation corresponding to the second mode in response to input of the first operational information after the n-th input of the second operational information, and activates operation corresponding to the first mode in response to input of the first operational information after the (n+1)-th input of the second operational information.

As described above, when the operating body Op is performing a pedaling operation to activate operation corresponding to one mode of the first and the second modes, a small movement thereof from the first operative unit 801 is sufficient to enable a pressing operation on the second operative unit 811b for causing operation to switch to the other mode. Additionally, when the operating body Op is performing a pressing operation to cause operation to switch to the other mode, a small movement thereof from the second operative unit 811b is sufficient to enable a pedaling operation on the first operative unit 801 for activating operation corresponding to the other mode. Thus, the pressing operation for causing operation to switch to the other mode and the pedaling operation for activating operation corresponding to the other mode can be performed easily and quickly while kicking off the second operation unit 72b and committing errors in operation are prevented.

When the fluoroscopy and the temporary-stop modes have been set as the first and the second modes, the system controller 90 activates X-ray emission for fluoroscopy in response to input of the first operational information before input of the second operational information, stops X-ray emission in response to the 1st input of the first operational information after input of the second operational information, and activates X-ray emission for fluoroscopy in response to the 2nd input of the first operational information after the 1st input of the first operational information is stopped. Alternatively, when information on first and second positions of the table 11 is set as the first and the second modes, the system controller 90 moves the table 11 to the first position in response to input of the first operational information before input of the second operational information, moves the table 11 to the second position in response to input of the first operational information after the n-th input of the second operational information, and moves the table 11 to the first position in response to input of the first operational information after the (n+1)-th input of the second operational information. Furthermore, when employing a biplane type, instead of a single-plane type formed of the X-ray irradiator 20 and the X-ray detector 26, the embodiment may be carried out as follows. The system controller 90 causes the first X-ray irradiator to emit X-rays in response to input of the first operational information before input of the second operational information, causes the second X-ray irradiator to emit X-rays in response to input of the first operational information after the (3m−2)-th input (m is a positive integer) of the second operational information, causes the first and the second X-ray irradiators to emit X-rays in response to input of the first operational information after the (3m−1)-th input of the second operational information, and causes the first irradiator to emit X-rays in response to input of the first operational information after the 3m-th input of the second operational information.

The system controller 90 stores: information on the first and the second modes set based on input from the first operation unit 71 in association with the first and the second operational information; and switching information for causing operation to switch from one mode to the other mode of the first and the second modes. The system controller 90 then controls the X-ray irradiator 20, the X-ray detector 26, the moving mechanism unit 31, the image processor 40, and the image data collector 50 on the basis of the information on the first and the second modes, the switching information, the first operational information, the second operational information, and the initialization information.

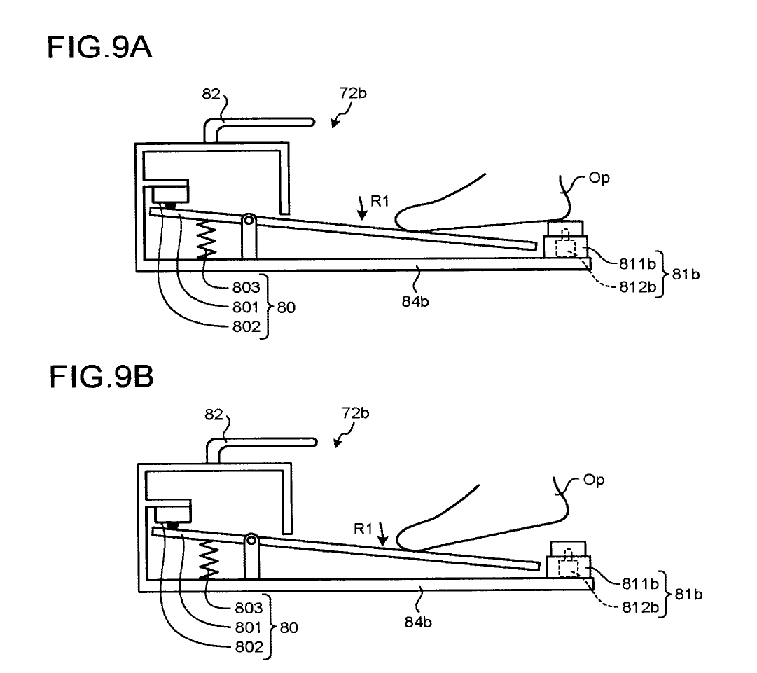

After the input of the initialization information, as illustrated in FIG. 9A, the system controller 90 activates operation corresponding to the first mode causing, for example, X-ray emission from the X-ray irradiator 20, in response to the (3m−2)-th input of the first and the second operational information by pedaling and pressing operations performed substantially concurrently on the first and the second operative units 801 and 811b.

The operating body Op can thus substantially concurrently perform pedaling and pressing operations on the first and the second operative units 801 and 811b as a result of positioning of the second operative unit 811b such that the operating body Op can touch the second operative unit 811b while touching the upper surface of the first operative unit 801.

Additionally, as illustrated in FIG. 9B, the system controller 90 activates operation corresponding to the second mode causing, for example, stopping of X-ray emission from the X-ray irradiator 20, in response to the (3m−1)-th input of the second operational information after the (3m−2)-th input of the second operational information is stopped with the (3m−2)-th input of the first operational information being continued, according to the following series of operations: after the (3m−2)-th input of the first and the second operational information, performing a releasing operation and then a pressing operation on the second operative unit 811b while continuously pedaling the first operative unit 801.

Thus, X-ray emission can be stopped with the pedaling and the pressing operations being continuously performed that do not force the operator into an uncomfortable posture.

Furthermore, the system controller 90 activates operation corresponding to the first mode in response to the 3m-th input of the second operational information after stopping of the (3m−1)-th input of the second operational information with the (3m−2)-th input of the first operational information being continued, according to the following series of operations: performing a releasing operation and then a pressing operation on the second operative unit 811b while continuously performing a pedaling operation on the first operative unit 801. The system controller 90 stops operation corresponding to the first and the second modes in response to stopping of input of the first operational information with input of the second operational information being stopped, or in response to stopping of input of the first and the second operational information according to releasing operations performed substantially concurrently.

As described above, when the operating body Op is performing pedaling and pressing operations to activate operation corresponding to one mode of the first and the second modes, a small movement thereof from the second operative unit 811b is sufficient to enable a releasing operation on the second operative unit 811b for causing operation to switch to the other mode with the first operative unit 801 being continuously pedaled. Additionally, when the operating body Op has performed a releasing operation to cause operation to switch to the other mode, a small movement thereof is sufficient to enable pedaling and pressing operations on the first and the second operative units 801 and 811b for activating operation corresponding to the other mode. Thus, the releasing operation for causing operation to switch to the other mode with a pedaling operation being continuously performed, and the pedaling and the pressing operations for executing operation corresponding to the other mode can be performed easily and quickly while kicking off the second operation unit 72b and committing errors in operation are prevented.

The system controller 90 activates operation corresponding to the first mode in response to the (3m−2)-th input of the first and the second operational information according to pedaling and pressing operations performed substantially concurrently on the first and the second operative units 801 and 811b. The system controller 90 activates operation corresponding to the second mode, in response to the (3m−1)-th input of the first operational information after the (3m−2)-th input of the first operational information is stopped while the (3m−2)-th input of the second operational information is continued, according to the following series of operations: after the (3m−2)-th input of the first and the second operational information, performing a releasing operation and then a pedaling operation on the first operative unit 801 while continuously performing a pressing operation on the second operative unit 811b. This embodiment may be further carried out in such a manner that the system controller 90 activates operation corresponding to the first mode in response to the 3m-th input of the first operational information after stopping of the (3m−1)-th input of the first operational information with the (3m−2)-th input of the second operational information being continued, in accordance with the following series of operations: performing a releasing operation and then a pedaling operation on the first operative unit 801 while continuously performing a pressing operation on the second operative unit 811b.

According to the third embodiment described above, the second operative unit 811b is positioned so that the operating body Op can touch the second operative unit 811b while touching the upper surface of the first operative unit 801. As a result of this positioning, when the operating body Op is performing a pedaling operation to activate operation corresponding to one of the first and the second modes, a small movement thereof from the first operative unit 801 is sufficient to enable a pressing operation on the second operative unit 811b for causing operation to switch to the other mode. Additionally, when the operating body Op is performing a pressing operation to cause operation to switch to the other mode, a small movement thereof from the second operative unit 811b is sufficient to enable a pressing operation on the first operative unit 801 for activating operation corresponding to the other mode.

Thus, the pressing operation for causing operation to switch to the other mode and the pedaling operation for activating operation corresponding to the other mode can be performed easily and quickly while kicking off the second operation unit 72b and committing errors in operation are prevented.

Furthermore, pedaling and pressing operations can be performed substantially concurrently on the first and the second operative units 801 and 811b to activate operation corresponding to one mode of the first and the second modes. When the operating body Op is performing pedaling and pressing operations to activate operation corresponding to the one mode, a small movement thereof from the second operative unit 811b is sufficient to enable a releasing operation on the second operative unit 811b for causing operation to switch to the other mode with the pedaling operation being continuously performed on the first operative unit 801. Additionally, when the operating body Op has performed the releasing operation for causing operation to switch to the other mode, a small movement thereof is sufficient to enable pedaling and pressing operations on the first and the second operative units 801 and 811b for activating operation corresponding to the other mode.

Thus, the pedaling and the pressing operations for activating operation corresponding to the one mode, the releasing operation for causing operation to switch to the other mode while continuously performing the pedaling operation, and the pedaling and the pressing operations for activating operation corresponding to the other mode can be performed easily and quickly while kicking off the second operation unit 72b and committing errors in operation are prevented.

### Fourth Embodiment

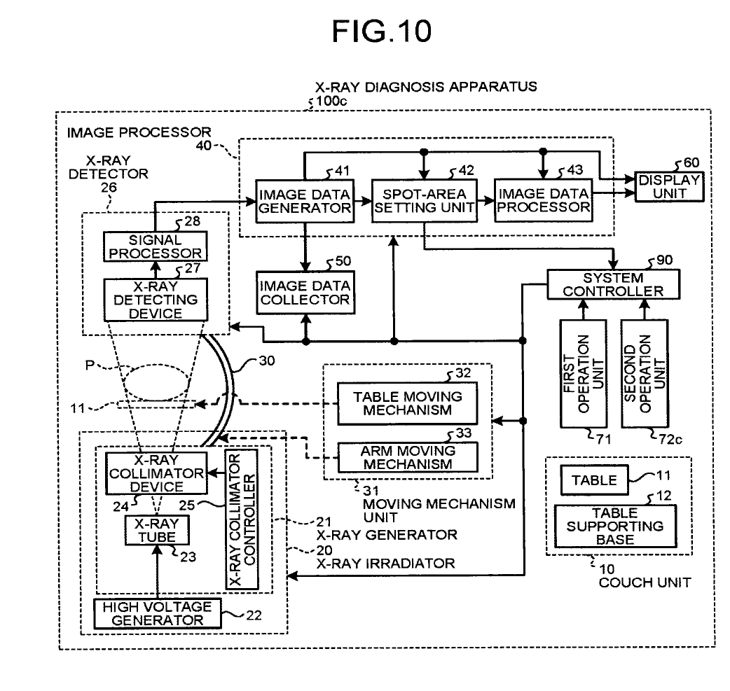

FIG. 10 is a block diagram illustrating the configuration of an X-ray diagnosis apparatus according to a fourth embodiment. This X-ray diagnosis apparatus 100c differs from the X-ray diagnosis apparatus 100b according to the third embodiment illustrated in FIG. 7 in that it includes a second operation unit 72c in place of the second operation unit 72b of the X-ray diagnosis apparatus 100b. Units included in the X-ray diagnosis apparatus 100c that have the same configurations and the same functions as those of the X-ray diagnosis apparatus 100b illustrated in FIG. 7 are assigned the same reference signs, and detailed descriptions thereof are omitted.

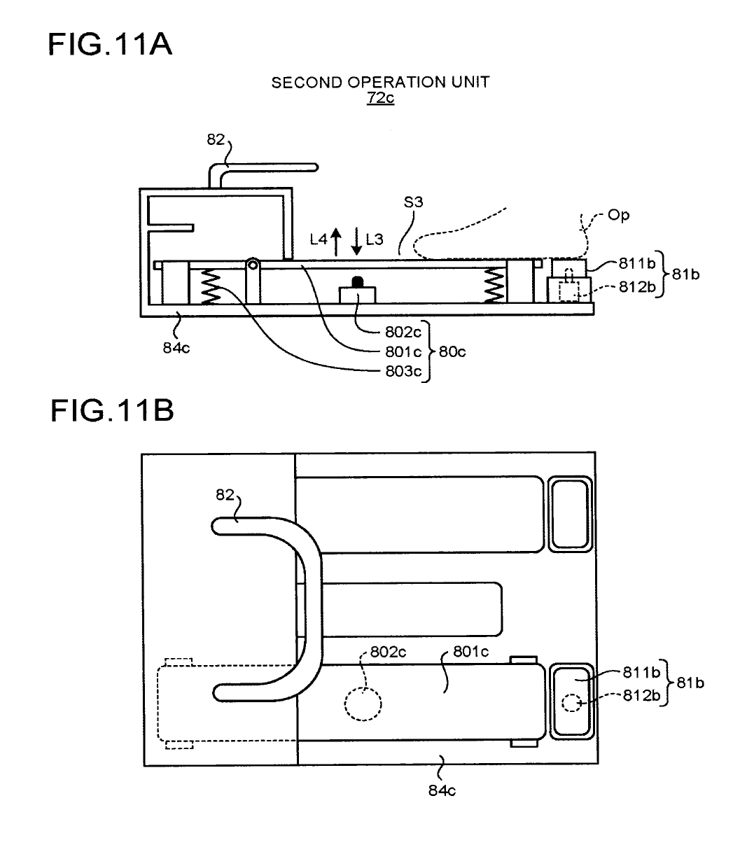

FIG. 11A and FIG. 11B are views illustrating the configuration of the second operation unit 72c. FIG. 11A is a side view of the second operation unit 72c. FIG. 11B is a top view of the second operation unit 72c.

This second operation unit 72c differs from the second operation unit 72b according to the third embodiment illustrated in FIGS. 8A and 8B in that the first operative unit is arranged so as to be movable parallel downward. Units included in the second operation unit 72c that have the same configurations and the same functions as those of the second operation unit 72b are assigned the same reference signs, and detailed descriptions thereof are omitted.

The second operation unit 72c includes input units including: at least one set of input units including a first input unit 80c for inputting first operational information, the second input unit 80b for inputting second operational information, and the third input unit 82 for inputting initialization information. The second operation unit 72b further includes a supporting body 84c supporting the respective input units.

The first input unit 80c includes a first operative unit 801c, a first switch 802c, and a spring 803c. The first operative unit 801c is arranged so that it can be pedaled by the operating body Op, and has a rectangular upper surface. The first switch 802c is turned on in response to a pedaling operation on the first operative unit 801c. The spring 803c expands and contracts in such a manner that allows the first operative unit 801c to be held at a stop position S3. The second input unit 81b is arranged near and apart from one end of the first operative unit 801c in the longitudinal direction thereof.

The first operative unit 801c is supported by parts of the supporting body 84c that are arranged near the four corners thereof, so that it can move parallel in a direction indicated by the arrow L3, that is, downward, from the stop position S3. When the first switch 802c is turned on in response to a pedaling operation performed on the first operative unit 801c by the operating body Op, the first operational information is input. When the first switch 802c is turned off in response to a releasing operation on the first operative unit 801c, input of the first operational information is stopped.

The second operative unit 811b is thus positioned so that the operating body Op can touch the second operative unit 811b while touching the upper surface of the first operative unit 801c. As a result of this positioning, a small movement of the operating body Op from one operative unit of the first operative unit 801c and the second operative unit 811b is sufficient to enable input of operational information corresponding to operation of the other.

The system controller 90 controls the X-ray irradiator 20, the X-ray detector 26, the moving mechanism unit 31, the image processor 40, and the image data collector 50 on the basis of: information on the first and the second modes, such as fluoroscopy and photography modes, fluoroscopy and temporary-stop modes, fluoroscopy and spot fluoroscopy modes, or fluoroscopy and fluoroscopy collection modes set in association with the first and the second operational information based on input from the first operation unit 71; switching information; first operational information; second operational information; and initialization information. The system controller 90 thus activates operation corresponding to the first mode or operation corresponding to the second mode in response to input of the first operational information or the second operational information, in the similar manner as in the first embodiment.

Here, when the fluoroscopy and the photography modes, the fluoroscopy and the spot fluoroscopy modes, the fluoroscopy and the fluoroscopy collection modes or the like have been set as the first and the second modes, the system controller 90 activates operation corresponding to the first mode in response to input of the first operational information before input of the second operational information after input of the initialization information, activates operation corresponding to the second mode in response to input of the first operational information after the n-th input of the second operational information, and activates operation corresponding to the first mode in response to input of the first operational information after the (n+1)-th input of the second operational information.

As described above, when the operating body Op is performing a pedaling operation to activate operation corresponding to one mode of the first and the second modes, a small movement thereof from the first operative unit 801c is sufficient to enable a pressing operation on the second operative unit 811b for causing operation to switch to the other mode. Additionally, when the operating body Op is performing a pressing operation to cause operation to switch to the other mode, a small movement thereof from the second operative unit 811b is sufficient to enable a pedaling operation on the first operative unit 801c for activating operation corresponding to the other mode. Thus, the pressing operation for causing operation to switch to the other mode and a pedaling operation for activating operation corresponding to the other mode can be performed easily and quickly while kicking off the second operation unit 72c and committing errors in operation are prevented.

Alternatively, when the fluoroscopy and the temporary-stop modes have been set, the system controller 90 activates X-ray emission for fluoroscopy in response to input of the first operational information before input of the second operational information, stops X-ray emission in response to the 1st input of the first operational information after input of the second operational information, and activates X-ray emission for fluoroscopy in response to the 2nd input of the first operational information after the 1st input of the first operational information is stopped. Further alternatively, when information on first and second positions of the table 11 has been set, the system controller 90 moves the table 11 to the first position in response to input of the first operational information before input of the second operational information, moves the table 11 to the second position in response to input of the first operational information after the n-th input of the second operational information, and moves the table 11 to the first position in response to input of the first operational information after the (n+1)-th input of the second operational information. Furthermore, when employing a biplane type instead of a single-plane type formed of the X-ray irradiator 20 and the X-ray detector 26 as explained above, the embodiment may be carried out as follows. The system controller 90 activates X-ray emission from the first X-ray irradiator in response to input of the first operational information before input of the second operational information, activates X-ray emission from the second X-ray irradiator in response to input of the first operational information after the (3m−2)-th input (m is a positive integer) of the second operational information, activates X-ray emission from the first and the second X-ray irradiators in response to input of the first operational information after the (3m−1)-th input of the second operational information, and activates X-ray emission from the first irradiator in response to input of the first operational information after the 3m-th input of the second operational information.

The system controller 90 controls the X-ray irradiator 20, the X-ray detector 26, the moving mechanism unit 31, the image processor 40, and the image data collector 50 on the basis of: the information on the first and the second modes set according to input from the first operation unit 71 in association with the first and the second operational information; the switching information; the first operational information; the second operational information; and the initialization information.

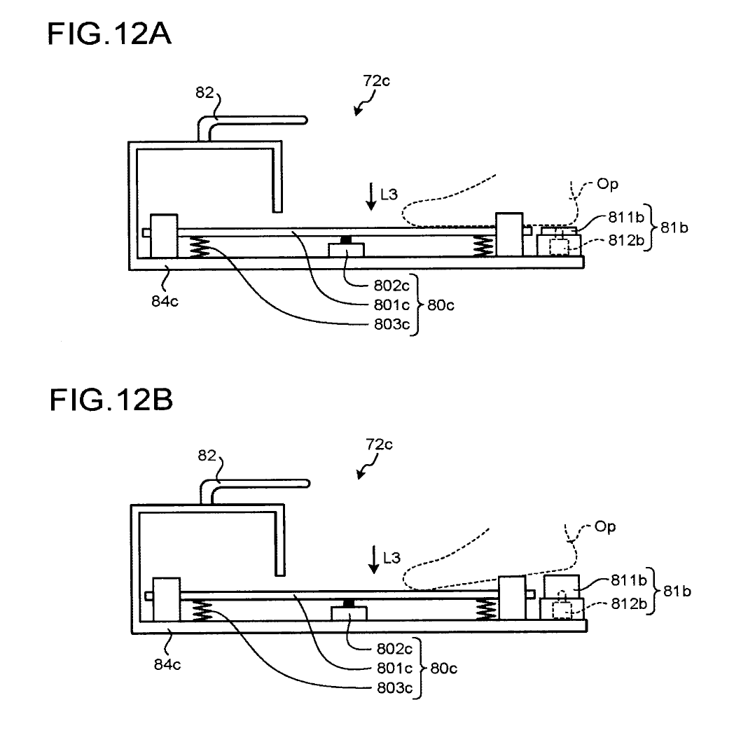

After input of the initialization information, as illustrated in FIG. 12A, the system controller 90 activates operation corresponding to the first mode causing, for example, X-ray emission from the X-ray irradiator 20, in response to the (3m−2)-th input (m is a positive integer) of the first and the second operational information with pedaling and pressing operations performed substantially concurrently on the first and the second operative units 801c and 811b.

The operating body Op can thus substantially concurrently perform pedaling and pressing operations on the first and the second operative units 801c and 811b as a result of positioning of the second operative unit 811b such that the operating body Op can touch the second operative unit 811b while touching the upper surface of the first operative unit 801c.

Additionally, as illustrated in FIG. 12B, the system controller 90 activates operation corresponding to the second mode causing, for example, stopping of X-ray emission from the X-ray irradiator 20, in response to the (3m−1)-th input of the second operational information after the (3m−2)-th input of the second operational information is stopped with the (3m−2)-th input of the first operational information being continued, according to the following series of operations: after the (3m−2)-th input of the first and the second operational information, performing a releasing operation and then a pressing operation on the second operative unit 811b while continuously pedaling the first operative unit 801c.

Thus, X-ray emission can be stopped with the pedaling and the pressing operations being continuously performed that do not force the operator into an uncomfortable posture.

Furthermore, the system controller 90 activates operation corresponding to the first mode in response to the 3m-th input of the second operational information after stopping of the (3m−1)-th input of the second operational information with the (3m−2)-th input of the first operational information being continued, according to the following series of operations: performing a releasing operation and then a pressing operation on the second operative unit 811b while continuously performing a pedaling operation on the first operative unit 601c. The system controller 90 stops operation corresponding to the first and the second modes in response to stopping of input of the first operational information with input of the second operational information being stopped, or in response to stopping of input of the first and the second operational information according to releasing operations performed substantially concurrently.

As described above, when the operating body Op is performing pedaling and pressing operations to activate operation corresponding to one mode of the first and the second modes, a small movement thereof from the second operative unit 811b is sufficient to enable a releasing operation on the second operative unit 811b for causing operation to switch to the other mode with the first operative unit 801c being continuously pedaled. Additionally, when the operating body Op has performed a releasing operation to cause operation to switch to the other mode, a small movement thereof is sufficient to enable pedaling and pressing operations on the first and the second operative units 801c and 811b for activating operation corresponding to the other mode. Thus, the releasing operation for causing operation to switch to the other mode, and the pedaling and the pressing operations for executing operation corresponding to the other mode can be performed easily and quickly while kicking off the second operation unit 72c and committing errors in operation are prevented.

According to the fourth embodiment described above, the second operative unit 811b is positioned so that the operating body Op can touch the second operative unit 811b while touching the upper surface of the first operative unit 801c. As a result of this positioning, when the operating body Op is performing a pedaling operation to activate operation corresponding to one of the first and the second modes, a small movement thereof from the first operative unit 801 is sufficient to enable a pressing operation on the second operative unit 811b for causing operation to switch to the other mode. Additionally, when the operating body Op is performing a pressing operation to cause operation to switch to the other mode, a small movement thereof from the second operative unit 811b is sufficient to enable a pressing operation on the first operative unit 801c for activating operation corresponding to the other mode.

Thus, the pressing operation for causing operation to switch to the other mode and the pedaling operation for activating operation corresponding to the other mode can be performed easily and quickly while kicking off the second operation unit 72c and committing errors in operation are prevented.

Furthermore, pedaling and pressing operations can be performed substantially concurrently on the first and the second operative units 801c and 811b to activate operation corresponding to one mode of the first and the second modes. When the operating body Op is performing pedaling and pressing operations to activate operation corresponding to the one mode, a small movement thereof from the second operative unit 811b is sufficient to enable a releasing operation on the second operative unit 811b for causing operation to switch to the other mode with the pedaling operation being continuously performed on the first operative unit 801c. Additionally, when the operating body Op has performed the releasing operation for causing operation to switch to the other mode, a small movement thereof is sufficient to enable pedaling and pressing operations on the first and the second operative units 801c and 811b for activating operation corresponding to the other mode.

Thus, the pedaling and the pressing operations for activating operation corresponding to the one mode, the releasing operation for causing operation to switch to the other mode while continuously performing the pedaling operation, and the pedaling and the pressing operations for activating operation corresponding to the other mode can be performed easily and quickly while kicking off the second operation unit 72c and committing errors in operation are prevented.

As described above, the first to the fourth embodiments enable easy operation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnosis apparatus comprising:
an X-ray irradiator that emits X-rays to a subject;
an X-ray detector that detects X-rays emitted from the X-ray irradiator and having passed through the subject;
a first input unit arranged in a manner that allows the first input unit to be pedaled, the first input unit being used to input first operational information in response to a pedaling operation performed thereon by an operator;
a second input unit arranged apart from the first input unit at a position that allows the operator to touch the second input unit while touching an upper surface of the first input unit that faces away from a floor surface of a room, the second input unit being used to input second operational information in response to an operation performed thereon by the operator; and
a controller that performs control for changing the first and the second operational information or either thereof on the basis of operational information input by at least one of the first and the second input units.

2. The X-ray diagnosis apparatus according to claim 1, wherein the second input unit is provided in a manner that allows the second input unit to be pressed in the longitudinal direction of the first input unit.

3. The X-ray diagnosis apparatus according to claim 1, wherein the second input unit is provided in a manner that allows the second input unit to be pressed by horizontally changing an angle of a foot of the operator that is placed on the upper surface of the first input unit.

4. The X-ray diagnosis apparatus according to claim 1, wherein
the first input unit is arranged in a manner that allows the first input unit to tilt downward or move parallel downward, and
the second input unit is used for inputting the second operational information in response to a pressing operation performed on the second input unit arranged near one end of the first input unit in the longitudinal direction thereof.

5. The X-ray diagnosis apparatus according to claim 1, further comprising a display unit that displays the first operational information.

6. The X-ray diagnosis apparatus according to claim 1, wherein, on the basis of the second operational information, the controller performs control by causing X-ray emission from the X-ray irradiator to switch between emission for fluoroscopy and for photography.

7. The X-ray diagnosis apparatus according to claim 1, wherein the controller activates operation corresponding to a first mode in response to input of the first operational information before input of the second operational information, activates operation corresponding to a second mode in response to input of the first operational information after the n-th input of the second operational information, and activates operation corresponding to the first mode in response to input of the first operational information after the (n+1)-th input of the second operational information, where n is a positive odd integer.

8. The X-ray diagnosis apparatus according to claim 7, further comprising a third input unit that is used to input initialization information, wherein
the controller causes operation to switch from the first mode to the second mode in response to the n-th input of the second operational information after input of the initialization information, and causes operation to switch from the second mode to the first mode in response to the (n+1)-th input of the second operational information, where n is a positive odd integer.

9. The X-ray diagnosis apparatus according to claim 7, wherein
the first mode is a fluoroscopy mode in which the X-ray irradiator emits X-rays for fluoroscopy, and
the second mode is a photography mode in which the X-ray irradiator emits X-rays for photography.

10. The X-ray diagnosis apparatus according to claim 7, wherein
the first mode is a fluoroscopy mode in which the X-ray irradiator emits X-rays for fluoroscopy to a given range, and
the second mode is a spot fluoroscopy mode in which the X-ray irradiator emits X-rays for fluoroscopy to a range corresponding to a spot area that is smaller than the given range.

11. The X-ray diagnosis apparatus according to claim 7, further comprising an image data collector that be controlled by the controller and collects image data generated as a result of emission of X-rays for fluoroscopy by the X-ray irradiator, wherein
the first mode is a fluoroscopy mode in which the X-ray irradiator emits X-rays for fluoroscopy, and
the second mode is a fluoroscopy collection mode in which the X-ray irradiator emits X-rays for fluoroscopy and the image data collector collects the image data.

12. The X-ray diagnosis apparatus according to claim 1, wherein the controller activates operation corresponding to the first mode in response to input of the first operational information after input of the second operational information, activates operation corresponding to the second mode in response to the first input of the first operational information after input of the second operational information, and activates operation corresponding to the first mode in response to the second input of the first operational information after the first input of the first operational information.

13. The X-ray diagnosis apparatus according to claim 1, wherein the controller activates operation corresponding to the first mode in response to the (3m−2)-th input of the first and the second operational information, activates operation corresponding to the second mode, with the (3m−2)-th input of the first or the second operational information being continued, in response to the (3m−1)-th input of the other operational information after the (3m−2)-th input of the other operational information is stopped, and activates operation corresponding to the first mode, with the (3m−2)-th input of the one operational information being still continued, in response to the 3m-th input of the other operational information after the (3m−1)-th input of the other operational information is stopped, where m is a positive integer.

14. The X-ray diagnosis apparatus according to claim 12, wherein
the first mode is a fluoroscopy mode in which the X-ray irradiator emits X-ray for fluoroscopy, and
the second mode is a temporary-stop mode in which the X-ray irradiator is stopped from emitting X-rays.

15. The X-ray diagnosis apparatus according to claim 1, wherein
the X-ray irradiator comprises first and second X-ray irradiators each that emit X-rays to the subject, and
the controller causes the first X-ray irradiator to emit X-rays in response to input of the first operational information before input of the second operational information, causes the second X-ray irradiator to emit X-rays in response to input of the first operational information after the (3m−2)-th input of the second operational information, causes the first and the second X-ray irradiators to emit X-rays in response to input of the first operational information after the (3m−1)-th input of the second operational information, and causes the first irradiator to emit X-rays in response to input of the first operational information after the 3m-th input of the second operational information, where m is a positive integer.
